(19) United States
(12) Patent Application Publication
(10) Pub. No.: US 2004/0057973 A1
(43) Pub. Date: Mar. 25, 2004

(54) Cosmetic preparations and use thereof, containing polyethylene waxes with improved organoleptic properties

(76) Inventors: Lars Wittkowski, Mannheim (DE); Katrin Zeitz, Ludwigshafen (DE); Wilhelm Weber, Neustadt (DE); Andreas Deckers, Flomborn (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/451,404

(22) PCT Filed: Dec. 21, 2001
(86) PCT No.: PCT/EP01/15239

(30) Foreign Application Priority Data
Dec. 22, 2000 (DE) 100 64 799.5
Dec. 22, 2000 (DE) 100 64 800.2

(51) Int. Cl. A61K 7/42; A61K 7/021; A61K 7/025; A61K 7/00

(52) U.S. Cl. 424/401; 424/59; 424/63; 424/64

(57) ABSTRACT

Cosmetic preparations comprising polyethylene waxes having improved organoleptic properties

Cosmetic preparations and toothpastes, comprising polyethylene waxes, in particular micronized polyethylene waxes with improved organoleptic properties prepared by (co)polymerization of ethylene under high-pressure conditions using an aliphatic or alicyclic ketone as molecular weight regulator.

The present invention relates to cosmetic preparations and applications, comprising polyethylene waxes with improved organoleptic properties prepared by (co)polymerization of ethylene under high-pressure conditions using an aliphatic or alicyclic ketone as molecular weight regulator, and to a process for the preparation of cosmetic preparations and applications having improved organoleptic properties. In addition, the present invention relates to the use of polyethylene waxes with improved organoleptic properties for cosmetic preparations and applications, for example for decorative cosmetics, care and cleansing cosmetics and pharmaceutical preparations or hygiene articles. In particular, the present invention relates to micronized polyethylene waxes prepared by (co)polymerization of ethylene under high-pressure conditions using an aliphatic or alicyclic ketone as molecular weight regulator and subsequent micronization, and to their use for cosmetic preparations and as carriers for toothpaste.

Polyethylene waxes are used in numerous cosmetics. The waxes to be used are subject to demanding requirements relating to their organoleptic properties. For example, the waxes must under no circumstances exude unpleasant odors because they would otherwise be uncommercial in cosmetic preparations. For the same reason, they must be flavorless. In addition, they must have good processing properties. Finally, they must be economically advantageous, i.e. it must be possible to prepare them easily and with high yield. Key parameters are the average molecular weight, molecular weight distribution and, in this connection in particular the breadth of the molecular weight distribution, and the high molecular weight and low molecular weight proportions, optionally incorporated comonomers, length and distribution of the branches and contamination of the polymer for example by volatile oligomers, residual monomers and decomposition products of catalysts or free-radical initiators and molecular weight regulators.

These parameters are particularly important in applications in which polyethylene waxes are used in micronized form. Micronized polyethylene waxes are understood below as meaning those waxes which have been converted to a suitable morphology by grinding or spraying, i.e. wax powder with a maximum particle diameter of 100 μm. A more recent application for the largely chemically inert polyethylene waxes are toothpastes, in which, being a carrier material with a less abrasive action, they can replace the otherwise added silica gel. Especially in modern toothpastes, recourse is frequently made to polyethylene wax as carrier instead of silica gel or calcium carbonate because polyethylene wax-containing toothpastes have a significantly weaker abrasive action and therefore the tooth enamel is not so greatly attacked, even with frequent cleaning, than in the case of the use of silica gel.

Polyethylene waxes can be prepared by various processes which can roughly be divided into low-pressure processes, carried out at 20 to 100 bar, and high-pressure processes, carried out at 500 to 4000 bar. The high-pressure process is a free-radical polymerization process which generally does not require a catalyst (cf., for example: Ullmann's Encyclopedia of Industrial Chemistry, 4th edition, key words: waxes, Vol. 24, p. 36 ff., Thieme Verlag Stuttgart, 1977). To start the free-radical chain reaction, use is made in most cases of one or more organic peroxides, for example the Trigonox® or Perkadox® trade names from Akzo Nobel, or else air or atmospheric oxygen. The cheapest and therefore the most widely used free-radical initiator is air or atmospheric oxygen.

To establish a suitable molecular weight, use is made of substances referred to as molecular weight regulators or, in short, regulators. Where a substance is used as regulator, it must be ensured that it is sufficiently efficient because the metered addition of very large amounts of regulators is uneconomical.

A frequently used regulator is hydrogen; however, where air or atmospheric oxygen is used as free-radical initiator, the hydrogen may lead to the formation of oxyhydrogen gas and therefore raises doubts from a safety viewpoint.

Other frequently used regulators are carbon monoxide CO and alkanes, such as, for example, ethane or propane. Carbon monoxide is very toxic, meaning that when it is used extensive safety measures are required. Regulators in gaseous form, such as ethane and propane, likewise require stringent safety precautions.

The frequently used aldehydes, for example propionaldehyde, lead to waxes which have a characteristic unpleasant odor and are only suitable as components for cosmetic applications, such as, for example, lipsticks, eyeshadows or blusher, after complex processing. Even after intense treatment with steam, it is still possible to detect a weak, but unpleasant odor.

The use of ketones as molecular weight regulators in the preparation of LDPE is already known. EP-A 0 928 797 proposes a process using methyl ethyl ketone as regulator, with the help of which an LDPE is prepared which is suitable for extrusion products, for example for films with good puncture resistance, but not for waxes.

DE-A 1 908 964 discloses a process by which ethylene homopolymers can be prepared in a high-pressure process. A characterizing feature of the process described is the use of a peroxidic free-radical initiator in the first reaction zone and air in the second. Recommended regulators are propionaldehyde or methyl ethyl ketone. This gives a high molecular weight polyethylene which is particularly suitable for the preparation of highly-transparent fine films or sturdy packaging films.

U.S. Pat. No. 3,334,081 describes a high-pressure polymerization process with increased conversion which is based on the feeding in of ethylene at at least two different sites of the reactor. Recommended as free-radical initiators are a large number of organic peroxides, and recommended as regulators are a large number of organic compounds, preferably ketones, such as, for example, methyl ethyl ketone. However, a disadvantage of the described process is the high investment expenditure, which is based on the numerous dosing sites, all of which have to be designed such that they are extremely pressure-stable and tight. As a result, the investment requirement for a polymerization plant is very high.

U.S. Pat. No. 3,317,504 discloses a process for the preparation of polyethylene suitable for the preparation of films, which requires a particular temperature control and the use of particular molecular weight regulators, for example methyl ethyl ketone. However, in the case of the process parameters disclosed, no waxes are obtained.

Romanian Patent RO 75,587 (priority: 04.18.1979, from CA 96: 200372s) describes the preparation of odorless LDPE grades. The regulator used is a mixture of methyl vinyl ketone, with propane, ethane and CO, and, to start the reaction, a mixture of different organic peroxides is used. However, because of the considerable toxicity, the use of CO is disadvantageous because the tubes and the reactor outlet have to be specially secured against the escape of CO.

In principle, polyethylene waxes can also be prepared in a low-pressure process. For this, a catalyst is generally required, for example a Ziegler-Natta catalyst as in U.S. Pat. No. 3,129,211, or a metallocene catalyst as in EP-A 0 890 619. The organoleptic properties of micronized polyethylene waxes prepared by a low-pressure process are, however, still in need of improvement in applications such as cosmetic preparations.

It is an object of the invention to provide polyethylene waxes in micronized or nonmicronized form for use in cosmetics, cosmetic preparations or toothpaste, which

can be prepared in an economically viable manner,

do not require the use of extremely explosion-hazardous regulators such as hydrogen or highly toxic regulators such as carbon monoxide,

have good organoleptic properties, i.e. are odorless and flavorless,

can be readily further processed

and are highly suitable for the preparation of cosmetic preparations.

We have found that these objects are achieved by using polyethylene waxes prepared with particular ketones.

The polyethylene waxes are prepared by polymerization or copolymerization of ethylene in high-pressure autoclaves or in tubular reactors. High-pressure autoclaves are known in compact or elongate variants. The known tubular reactors (Ullmann's Encyclopedia of Industrial Chemistry, Volume 19, p. 169 and p. 173 ff (1980), Verlag Chemie Weinheim, Deerfield Beach, Basle and Ullmann's Encyclopedia of Industrial Chemistry, 4th edition, key words: waxes, Vol. 24, p. 36 ff., Thieme Verlag Stuttgart, 1977) are characterized by simple handling and low maintenance and are advantageous compared with stirred autoclaves. The polymerization or copolymerization is usually carried out at temperatures of from 400 to 4000 bar, preferably from 500 to 5000 bar and particularly preferably 1000 to 3500 bar.

The reaction temperature is 180 to 350° C., preferably 200 to 320° C. It is also possible to prepare copolymers with ethylene, in which case all olefins which are free-radically copolymerizable with ethylene are in principle suitable as comonomers. Preference is given to

1-olefins, such as propylene, 1-butene, 1-pentene, 1-hexene, 1-octene and 1-decene,

acrylates, such as acrylic acid, methyl acrylate, ethyl acrylate, n-butyl acrylate, 2-ethylhexyl acrylate or tert-butyl acrylate;

methacrylic acid, methyl methacrylate, ethyl methacrylate, n-butyl methacrylate or tert-butyl methacrylate;

vinyl carboxylates, particular preference being given to vinyl acetate,

unsaturated dicarboxylic acids, particularly preferably maleic acid,

unsaturated dicarboxylic acid derivatives, particularly preferably maleic anhydride and alkylimides of maleic acid, e.g. N-methylmaleimide.

The comonomer fraction is at most 50 mol %, preferably at most 20 mol %.

We have found that the above-described polyethylene waxes with improved organoleptic properties can best be prepared by using one or more aliphatic or alicyclic ketones of the formula I

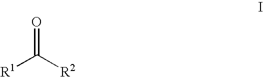

as regulator during the polymerization

Here, the radicals R¹ and R² are identical or different and chosen from

C₁-C₆-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, particularly preferably C₁-C₄-alkyl, such as methyl, ethyl, n-propyl, isopropyl, n-butyl, isobutyl, sec-butyl and tert-butyl;

C₃-C₁₂-cycloalkyl, such as cyclopropyl, cyclobutyl, cyclopentyl, cyclohexyl, cycloheptyl, cyclooctyl, cyclononyl, cyclodecyl, cycloundecyl and cyclododecyl; preferably cyclopentyl, cyclohexyl and cycloheptyl;

In a further embodiment, the radicals R¹ and R² are covalently bonded to one another. Thus, R¹ and R², for example, may be combined: —(CH₂)₄—, —(CH₂)₅—, —(CH₂)₆, —(CH₂)₇—, —CH(CH₃)—CH₂—CH₂—CH(CH₃)— or —CH(CH₃)—CH₂—CH₂—CH₂—CH(CH₃)—.

Preferred examples are acetone, methyl ethyl ketone “MEK”, methyl isobutyl ketone “MIBK”, 2-pentanone, 3-pentanone or cyclopentanone, cyclohexanone or cycloheptanone. Particularly preferred examples are acetone, methyl ethyl ketone, methyl isobutyl ketone and cyclohexanone; very particularly preferred are methyl ethyl ketone and, especially, cyclohexanone. Under these conditions, it is possible to dispense with the use of CO, ethane and propane as regulator.

After the preparation, the wax, which is normally produced as granules, grit or powders in industrial plants, can be used immediately in the corresponding cosmetic applications.

The above-described polyethylene waxes have a density of from 0.8 to 1.0 g/cm³, preferably from 0.90 to 0.96 g/cm³ and particularly preferably from 0.93 to 0.95 g/cm³, measured at 23° C. The melt viscosities are in the range from 20 to 20 000 cSt, preferably in the range from 800 to 2000 cSt, measured at 120° C., which corresponds to a molecular weight M_{w} of at most 40 000 g, preferably at most 10 000 g and particularly preferably at most 7500 g. The molecular weight distribution is in the range from 2 to 10. The melting points are in the range from 60 to 125° C., preferably 80 to 120° C.

The waxes exhibit very good organoleptic properties, in particular they are odorless and flavorless. This is surprising since both the ketones used and, in particular, methyl ethyl ketone and also the dimers, trimers and similar products which are readily formed therefrom under high-pressure conditions have a characteristic and by no means pleasant odor of their own.

The measurement of the organoleptic properties can be determined by means of apparatus, for example by gas chromatography or differential thermogravimetry, in which case the amount and the type of volatile compounds which are escaping is determined by separate or serially connected measurement apparatuses. Of great significance are the tests by test panels.

Because of their very good organoleptic properties, the above-described waxes are suitable in particular for preparations and applications of decorative cosmetics, chosen from lipsticks, face powder, blushers, eyeshadows, eyeliner pencils, foundations, make-up preparations, mascara and eyebrow pencils. The present invention therefore provides the use of the described polyethylene waxes for preparations and applications of decorative cosmetics, chosen from lipsticks, face powder, blushers, eyeshadows, eyeliner pencils, foundations, make-up preparations, mascara and eyebrow pencils.

The present invention further provides the use of the above-described waxes for preparations and applications of care and cleansing cosmetics, chosen from cleansing lotions, handcreams, handwashing pastes, skincare compositions, intimate care compositions, footcare compositions, sunscreen compositions, repellents, shaving compositions, hair-removal compositions, antiacne compositions, make-up, nailcare compositions, kohl pencils, soaps, syndets, liquid washing, showering and bath preparations, cold creams, skincare products, exfoliating scrub soaps, face, hand or foot peels, body scrubs, gels and lotions.

The above-described polyethylene waxes can be formulated readily and thus converted into the application form required or highly suitable for use. For example, different particle sizes are obtainable, for example, by spraying.

In addition, the waxes can be granulated in very diverse ways. The methods of granulation of polyethylene waxes are known per se. The above-described waxes can be granulated particularly well.

To prepare the abovementioned preparations of decorative cosmetics and the care and cleansing cosmetics and the pharmaceutical preparations and hygiene articles, for example incontinence products, the processes used are those known per se and familiar to the person skilled in the art. They do not have to be adapted compared with the prior art.

The present invention further provides preparations and applications of decorative cosmetics, chosen from lipsticks, face powder, blushers, eyeshadows, eyeliner pencils, foundations, make-up preparations, mascara and eyebrow pencils comprising the above-described polyethylene waxes with good organoleptic properties.

The present invention further provides preparations and applications of care and cleansing cosmetics, chosen from cleansing lotions, handcreams, hand washing pastes, skin care compositions, personal hygiene care compositions, foot care compositions, light protection compositions, repellents, shaving compositions, depilatories, antiacne compositions, make-up, nail care compositions, kohl pencils, soaps, syndets, liquid washing, shower and bath preparations, cold creams, skin care products, exfoliating scrub soaps, face, hand or foot peelings, body scrubs, gels and lotions, comprising the above-described polyethylene waxes with improved organoleptic properties.

The cosmetic preparations and applications according to the invention may, for example, be a solution, an anhydrous preparation, an emulsion or microemulsion of the water-in-oil type or of the oil-in-water type, a multiple emulsion, for example of the water-in-oil-in-water type, a gel, a solid stick, an ointment, an aerosol or else an aqueous system or a surfactant preparation for cleansing skin and/or hair. They comprise the above-described waxes in an amount of from 0.5 to 20% by weight.

In addition to the above-described polyethylene waxes, the cosmetic preparations and applications according to the invention may comprise: active ingredients, such as, for example, UV filters, perfumes and fragrances, emulsifiers, surfactants, solid carrier materials, such as, for example, chalk, further waxes, such as, for example, beeswax or carnauba waxes, vitamins, bases or acids, alcohols and water. In this connection, for various preparations it may be advantageous to use two or more different emulsifiers, active ingredients, surfactants, solid carrier materials, perfumes and fragrances, waxes, bases or acids, vitamins or alcohols.

The cosmetic preparations and applications according to the invention are prepared by methods known per se. Preference is given to mixing the components by stirring. Mixing may be carried out by stirring all of the required components in one step or consecutively in two or more steps. In this connection, the process can be carried out at room temperature or else at elevated temperatures to above the melting point of the above-described polyethylene waxes. For some preparations such as, for example, lipsticks, mixing at temperatures above the melting point of the polyethylene waxes is preferred. The maximum appropriate temperature for the mixing of the components is 150° C.

In the case of the preparation of those applications for which a spherical form of the polyethylene wax is required, the mixing is carried out at temperatures below the melting point of the polyethylene wax.

A particular embodiment of the present invention covers micronized polyethylene waxes with improved organoleptic properties, and a process for the preparation of micronized polyethylene waxes. Following the polymerization, the wax, which is usually produced as grit in industrial plants, is micronized according to the invention in a subsequent step. For the micronization, mills can be used, for example air-jet mills. However, the waxes can also be dissolved or melted and be sprayed in a spray tower. As the result of the micronization, the wax is produced as so-called micropowder or micronizate, where the particle diameter does not exceed 100 μm (median), preferably 50 μm and particularly preferably 30 μm.

The micronized polyethylene waxes according to the invention have a density of from 0.8 to 1.0 g/cm³, preferably from 0.90 to 0.96 g/cm³ and particularly preferably from 0.93 to 0.95 g/cm³, measured at 23° C. The melt viscosities are in the range from 20 to 20 000 cSt, preferably in the range from 800 to 2000 cSt, measured at 120° C., this corresponds to a molecular weight M_{w} of at most 40 000 g, preferably at most 10 000 g and particularly preferably at most 7500 g. The molecular weight distribution is in the range from 2 to 10. The melting points are in the range from 60 to 125° C., preferably 80 to 120° C. The maximum particle diameter is at most 30 μm following the micronization, which has been achieved by spraying or by grinding.

Both before and also after the micronization, the waxes exhibit very good organoleptic properties, in particular, they are odorless and flavorless. This is surprising since both the ketones used and, in particular, methyl ethyl ketone, and also the dimers, trimers and similar products which readily form therefrom under high-pressure conditions have a characteristic and in no way pleasant intrinsic odor.

The measurement of the organoleptic properties can be determined by means of apparatus, for example by gas chromatography or differential thermogravimetry, in which case the amount and the type of volatile compounds which are escaping is determined by separate or serially connected measurement apparatuses. Of great significance are the tests by test panels, in particular in the case of micronizates.

Because of their very good organoleptic properties, the micronizates according to the invention are particularly suitable for use as carrier materials or abrasives for toothpaste or cosmetic applications, such as, for example, lipsticks, blusher, eyeshadows and creams, such as, for example, hand creams or face creams.

The invention is illustrated by working examples.

### WORKING EXAMPLE

A) Preparation of the Polymers

Ethylene was polymerized in the presence of the respective molecular weight regulator (Examples No. 1 to 18) in a high-pressure autoclave, as described in the literature (M. Buback et al., Chem. Ing. Tech. 1994, 66, 510.). Monomer or monomer mixture to which tert-butyl peroxide and tert-butyl peroxypivalate had been added as initiator, and also the regulator were fed into this under the reaction pressure of 1700 bar. Table 1 gives the polymerization conditions and analytical data of the resulting polymers. In all of the experiments, a reaction temperature of 220° C. and a reaction pressure of 1700 bar were established.

TABLE 1
Polymerization conditions for Examples 1-9 and
Comparative Examples V1-V9.

| | Reg- | | | | | | |
|---|---|---|---|---|---|---|---|
| | ulator | Reg- | Ethyl- | Yield | | | |
| | (sub- | ulator | ene | [kg | r | h | Tₘₑₗₜ |
| No. | stance) | [l/h] | [kg/h] | PE/h] | [g/cm³] | mm²/s | ° C. |
| 1 | MEK | 2.35 | 12.2 | 2.0 | 0.9350 | 1080 | 112.8 |
| 2 | MEK | 2.70 | 12.1 | 1.8 | 0.9345 | 1880 | 112.9 |
| 3 | MEK | 2.65 | 12.0 | 1.9 | 0.9332 | 4110 | 113.0 |
| 4 | MIBK | 3.65 | 11.3 | 2.0 | 0.9348 | 870 | 110.6 |
| 5 | MIBK | 2.85 | 11.6 | 2.0 | 0.9344 | 1930 | 112.0 |
| 6 | MIBK | 2.15 | 12.2 | 1.9 | 0.9344 | 6530 | 112.0 |
| 7 | Acetone | 3.25 | 12.0 | 2.2 | 0.9340 | 1180 | 112.3 |
| 8 | Acetone | 2.70 | 12.3 | 2.2 | 0.9339 | 1830 | 112.9 |
| 9 | Acetone | 2.10 | 12.1 | 2.0 | 0.9328 | 4530 | 112.8 |
| V1 | PA | 0.34 | 11.6 | 1.5 | 0.9414 | 850 | 113.7 |
| V2 | PA | 0.24 | 11.4 | 1.5 | 0.9379 | 2070 | 113.6 |
| V3 | PA | 0.20 | 11.1 | 1.7 | 0.9372 | 4390 | 114.5 |
| V4 | IVA | 0.45 | 11.7 | 1.5 | 0.9256 | 4980 | 110.5 |
| V5 | IVA | 0.38 | 12.1 | 1.6 | 0.9245 | 2110 | 109.6 |
| V6 | IVA | 0.34 | 12.1 | 1.7 | 0.9254 | 4320 | 110.5 |
| V7 | C7 | 5.80 | 12.1 | 2.2 | 0.9256 | 4980 | 110.5 |
| V8 | C7 | 7.60 | 12.4 | 2.4 | 0.9245 | 2110 | 109.6 |
| V9 | C7 | 6.25 | 12.4 | 2.3 | 0.9254 | 4320 | 110.5 |

Abbreviations used:
MEK methyl ethyl ketone, MIBK methyl isobutyl ketone, PA: propionaldehyde, IVA: isovaleraldehyde, C7: n-heptane; PE: polyethylene wax.

Abbreviations used: MEK methyl ethyl ketone, MIBK methyl isobutyl ketone, PA: propionaldehyde, IVA: isovaleraldehyde, C7: n-heptane; PE: polyethylene wax.

B) Organoleptic testing of the polymers

To test the waxes from the above-listed Examples 1 to 9 and V1 to V9, the samples were in each case placed in front of two groups of test-persons. Test panel 1 consisted of 23 people without particular preparatory training. Test panel 2 consisted of 12 people who carry out odor and taste tests professionally. The assessment was carried out in each case using grades (1: very good, 2: good, 3: satisfactory, 4: adequate, 5: inadequate) and is given in Table 2.

TABLE 2
Organoleptic testing

| Sample | Test panel 1 | Test panel 2 | Regulator used |
|---|---|---|---|
| 1 | 1 | 1 | MEK |
| 2 | 1 | 1-2 | MEK |
| 4 | 1-2 | 2 | MIBK |
| 5 | 2 | 1 | MIBK |
| 7 | 2 | 2 | Acetone |
| 8 | 2-3 | 2-3 | Acetone |
| V1 | 3, 5 | 5 | PA |
| V2 | 4 | 4 | PA |
| V4 | 3 | 3, 5 | IVA |
| V5 | 4 | 3, 5 | IVA |
| V7 | 3-4 | 3-4 | C7 |
| V8 | 3-4 | 3 | C7 |

The described samples 1 to 9 and V1 to V9 were incorporated into peeling gels, and a lipstick was prepared in accordance with a standard procedure (at elevated temperature, i.e. with melting of the wax). The peeling gels prepared using the described samples 1 to 9 were significantly superior to the comparison peeling gels with regard to odor, with peeling gels 1 and 2 coming out best.

C) Application Examples

C.1 Example of the Formulation of a Peeling Gel:

| % by wt. | Component |
|---|---|
| 0.5 | vitamin E acetate |
| 3.0 | Cremophor RH 410 |
| 15.0 | ethanol |
| 0.01 | Uvinul ® D 50 |
| 3.0 | D-Panthenol USP |
| 0.85 | Neutrol ® TE |
| 60.0 | Carbopol ® 940 (1% strength) |
| 5.0 | polyethylene wax from Experiment 1 |

The peeling gel described was tested by the test panels and given a score of 1-2. An analogously prepared peeling gel in which polyethylene wax from Experiment V1 was used was given a score by the test panels of 4 and 5.

C.2 Example of the Formulation of a Lipstick:

In lipsticks prepared using samples 1 to 9, the perfume was significantly more effective than in the comparison samples which had been prepared using samples V1 to V9.

Lipstick: The components were mixed at elevated temperature, i.e. with melting of the wax components.

| % by wt. | Component |
|---|---|
| 3.0 | carnauba wax |
| 4.0 | candelilla wax |
| 2.0 | beeswax |
| 7.0 | microcrystalline wax (paraffin wax) with a |
| | melting point 52-54° C. |
| 1.5 | cetyl palmitate |
| 6.0 | petrolatum white |
| 4.0 | lanolin wax |
| 11.0 | cetearyl octanoate |
| 2.0 | polyethylene |
| 0.2 | bisabolol |
| 6.0 | hydrogenated cocoglycerides |
| 2.0 | tocopheryl acetate |
| 0.5 | tocopherol |
| 50.8 | castor oil |

C.3. Hand Cream Formulation Example:

Hand cleanser with exfoliating substances (all numerical values in percentages by weight):

Sulfopon® HC granules 13.70

| | |
|---|---|
| Glucopon ® 650 EC | 1.00 |
| Dehyton ® K | 1.2 |
| Texapon ® NSO | 1.00 |
| Comperlan ® KD | 0.50 |
| Chalk | 32.40 |
| Polyethylene wax | 8.10 |
| Soda | 0.90 |
| Edenor ® HT 35 | 1.10 |
| NaOH (50% strength) | 0.30 |
| Citric acid | 3.00 |
| Perfume oil | 0.30 |
| Dem. water | make up to 100 |

pH: 5.5-6.0

A variety of hand cleansers containing the polyethylene wax samples 1, 4, 5, V1 and V9 were prepared and assessed by the test panels as follows:

| Wax used | Test panel 1 | Test panel 2 |
|---|---|---|
| 1 | 1-2 | 1 |
| 4 | 3 | 3 |
| 5 | 3 | 3-4 |
| V1 | 4-5 | 5 |
| V9 | 5 | 5 |

D) Preparation and Organoleptic Testing of the Micronizates

The polymers produced in A) were micronized after the polymerization. The micronization was carried out each time on an air-jet mill from Micro-Macinazione, model Chrispro-Jetmill MC 100, to a particle size of 10 μm (median). Micronizates were obtained.

To test the waxes from the above-described examples 1 to 9 and V1 to V9, the samples were placed in front of the test panels from A). The assessment is given in Table 3.

TABLE 3
Organoleptic testing of the micronizates

| Sample | Test panel 1 | Test panel 2 | Regulator used |
|---|---|---|---|
| 1 | 1 | 1 | MEK |
| 2 | 1 | 1-2 | MEK |
| 4 | 1-2 | 2 | MIBK |
| 5 | 2 | 1 | MIBK |
| 7 | 2 | 2 | Acetone |
| 8 | 2-3 | 2-3 | Acetone |
| V1 | 3, 5 | 5 | PA |
| V2 | 4 | 4 | PA |
| V4 | 3 | 3, 5 | IVA |
| V5 | 4 | 3, 5 | IVA |
| V7 | 3-4 | 3-4 | C7 |
| V8 | 3-4 | 3 | C7 |

The described samples 1 to 9 and V1 to V9 were incorporated into toothpastes, and a cosmetic soap was prepared in accordance with a standard precedure. The toothpastes prepared using the described samples 1 to 9 were significantly superior to the comparison toothpastes with regard to odor, with pastes 1 and 2 coming out best.

Formulation of a toothpaste:

| Constituents (data in % by | | |
|---|---|---|
| weight) | | |
| (INCI name) | Example A | Example B |
| Water | 39.7 | 39.7 |
| Sorbitol | 35.0 | 35.0 |
| Hydrous silica gel | 0.0 | 0.0 |
| Luvitol ® PE 10 M | 20.0 | 20.0 |
| Sodium | 1.6 | 1.6 |
| carboxymethylcellulose | | |
| Saccharine | 0.2 | 0.2 |
| TiO₂ | 0.5 | 0.5 |
| Sodium monofluorophosphate | 1.0 | 1.0 |
| Aroma oil | 0.5 | 0.5 |
| Silica gel | 1.5 | 1.5 |

All of the constituents were homogenized at room temperature.

Example A was prepared using micronized wax, sample 1, and Example B was prepared using sample V2. Whereas toothpaste (Example A) exuded a refreshing odor, toothpaste of Example B had an odor component which a number of members of test panel 1 perceived as extremely unpleasant.

In lipsticks prepared using samples 1 to 9, the perfuming was significantly more effective than in the comparison samples prepared using samples V1 to V9.

Example of the formulation of a lipstick:

Lipstick

| % by wt. | Component |
|---|---|
| 3.0 | carnauba wax |
| 4.0 | candelilla wax |
| 2.0 | beeswax |
| 7.0 | microcrystalline wax (paraffin wax) with a |
| | melting point 52-54° C. |
| 1.5 | cetyl palmitate |
| 6.0 | petrolatum white |
| 4.0 | lanolin wax |
| 11.0 | cetearyl octanoate |
| 2.0 | micronized polyethylene |
| 0.2 | bisabolol |
| 6.0 | hydrogenated cocoglycerides |
| 2.0 | tocopheryl acetate |
| 0.5 | tocopherol |
| 50.8 | castor oil |

The polyethylene waxes prepared with ketones, in particular with methyl ethyl ketone or cyclohexanone as regulator are likewise of primary suitability for odorless peeling gels. Example of the formulation of a peeling gel:

1. A cosmetic preparation, comprising polyethylene waxes prepared by (co)polymerization of ethylene at pressures from 400 to 4000 bar and temperatures in the range from 180 to 350° C. using one or more aliphatic or alicyclic ketones as molecular weight regulator.

2. A cosmetic preparation as claimed in claim 1, comprising polyethylene waxes with a maximum molecular weight M_{w} of 40 000 g.

3. A preparation of decorative cosmetics, chosen from lipsticks, face powders, blushers, eyeshadows, eyeliner pencils, foundations, make-up preparations, mascara and eyebrow pencils, as claimed in claims 1 and 2.

4. A preparation of care and cleansing cosmetics chosen from cleansing lotions, handcreams, handwashing pastes, skincare compositions, intimate care compositions, footcare compositions, sunscreen compositions, repellents, shaving compositions, hair-removal compositions, antiacne compositions, make-up, nailcare compositions, kohl pencils, soaps, syndets, liquid washing, showering and bath preparations, cold creams, skincare products, exfoliating scrub soaps, face, hand or foot peels, body scrubs, gels and lotions, as claimed in claims 1 and 2.

5. A preparation for pharmaceutical preparations or hygiene articles as claimed in claims 1 and 2.

6. A process for the preparation of cosmetic preparations as claimed in claims 1 to 5, which comprises mixing 0.5 to 20% by weight of polyethylene wax at 20 to 200° C. with further substances chosen from active ingredients, such as, for example, UV filters, perfumes and fragrances, emulsifiers, surfactants, solid carrer materials, such as, for example, chalk, further waxes, such as, for example, beeswax or carnauba waxes, vitamins, bases or acids, alcohols and water.

7. The use of polyethylene waxes as claimed in claims 1 and 2 for cosmetic preparations.

8. A micronized polyethylene wax with a particle diameter (medium valve) of at most 100 μm, prepared by (co)polymerization of ethylene under high-pressure conditions using one or more aliphatic or alicyclic ketones as molecular weight regulator and subsequent micronization.

9. A micronized polyethylene wax as claimed in claim 8, prepared using methyl ethyl ketone or cyclohexanone as molecular weight regulator.

10. A micronized polyethylene wax as claimed in claims 8 and 9 with a maximum molecular weight M_{w} of 40 000 g.

11. A micronized polyethylene wax as claimed in claims 8 to 10 with a maximum particle diameter of 30 μm.

12. The use of micronized polyethylene waxes as claimed in claims 8 to 11 for cosmetic preparations and as carriers for toothpaste.

13. The use of micronized polyethylene waxes as claimed in claims 8 to 12 for eyeshadows, lip pencils or blusher.
